(19) United States
(12) Patent Application Publication
(10) Pub. No.: US 2011/0125204 A1
(43) Pub. Date: May 26, 2011

(54) Electrostimulation pad with integrated temperature sensor

(76) Inventor: Mohn Louise, London (GB)

(21) Appl. No.: 12/592,492

(22) Filed: Nov. 25, 2009

(51) Int. Cl. A61N 1/00; A61F 7/08

(52) U.S. Cl. 607/3; 607/96

(57) ABSTRACT

A pad including a heating element and a temperature sensor operationally coupled to sense thermal energy within the pad in close proximity to the heating element. The temperature sensor is a film type resistance thermal detector. The pad includes at least one contact, preferably two contacts, in a parallel relation with the heating element for providing electrical stimulation. The heating element, the first and second electrical contacts and the sensor are encapsulated in a single unitary multilayer injection molded construction. The invention enables new thermal and thermostimulation methods and protocols by which the electrical stimulation and/or the heating current supplied to a thermo and/or thermostimulation pad are adjusted based on the temperature sensed by the pad per se.

### BACKGROUND OF THE INVENTION

1. Field of the Invention

The present invention relates to therapeutic systems. More specifically, the present invention relates to systems and methods for sensing thermal energy generated by heating and/or electrostimulation pads.

2. Description of the Related Art

Pads are used in a variety of therapeutic treatment modalities currently known in the art including electrical stimulation, heat therapy and thermostimulation. Electrical stimulation involves the application of an electrical current to a single muscle or a group of muscles. The resulting contraction can produce a variety of effects from strengthening injured muscles and reducing oedema to relieving pain and promoting healing. Typical electrical stimulation systems are limited to two to four channels and therefore allow only two to four pads to be applied to a patient. The pads are usually quite small and often powered with a battery. This results in the application of a small amount of power and a low treatment depth of the resulting electric field. The shallow depth of the electric field generated by conventional electrical stimulation systems limits performance and patient benefit. Some systems have attempted to address this limitation by applying more current, often from a line or main supply source. However, the small size of conventional electrical stimulation pads is such that on the application of larger amounts of power, i.e. the use of higher currents, patients often report the experience of pain or discomfort.

Heat therapy or thermal stimulation itself is very useful as it has a number of effects such as relaxation of muscle spasm and increased blood flow that promotes healing. However, combination therapy, i.e. the synergistic use of other modalities such as massage, ultrasound and/or electrical stimulation has been found to be more effective than heat therapy alone.

Thermostimulation is one such combination therapy that involves the use of heat therapy and electrical stimulation simultaneously. With thermostimulation, the healing benefits of heat are provided along with the strengthening, toning, pain relieving and healing benefits of electrical stimulation. Moreover, the application of heat has been found effective in that it allows the patient to tolerate higher currents. This yields higher electric fields strengths, greater depths of penetration and therefore, more positive results than could be achieved with electrical stimulation without heat.

Unfortunately, conventional thermostimulation pads do not include heat sensors. As a consequence, the associated control systems and protocols must be limited to protect the patient from excessive and deleterious high temperatures. Moreover, without temperature sensing and feedback, more sophisticated treatment modalities are not possible with convention thermostimulation pads.

Hence, a need exists in the art for an improved thermostimulation pad design with an integrated temperature sensing capability.

### SUMMARY OF THE INVENTION

The need in the art is addressed by the therapeutic pad of the present invention. Generally, the pad includes a heating element and a temperature sensor operationally coupled to sense thermal energy within the pad in close proximity to the heating element. In the illustrative embodiment, the temperature sensor is a film type resistance thermal detector. Preferably, the pad includes at least one contact, preferably two contacts, in a parallel relation with the heating element for providing electrical stimulation. The heating element, the first and second electrical contacts and the sensor are encapsulated in a single unitary multilayer injection molded construction. The present invention enables new thermal and thermostimulation methods and protocols by which the electrical stimulation and/or the heating current supplied to a thermo and/or thermostimulation pad are adjusted based on the temperature sensed by the pad per se.

### BRIEF DESCRIPTION OF THE DRAWINGS

FIG. 1 is a simplified perspective view of a thermostimulation system implemented in accordance with an illustrative embodiment of the present teachings.

FIG. 2 shows a perspective bottom view of the pad of FIG. 1.

FIG. 3 is an exploded upside down view of a portion of the pad of FIG. 2 in disassembled relation.

FIG. 4 is a magnified perspective view of a heat sensor depicted FIG. 3 implemented as a conventional resistance temperature detector (RTD).

FIG. 5 is a sectional side view of an illustrative implementation of the RTD sensor depicted in FIG. 4.

FIG. 6 is a top plan view of the heating element of the illustrative embodiment of the pad of FIG. 1.

FIG. 7 is a magnified view of a portion of the heating element of FIG. 6.

FIG. 8 is a sectional side view of the pad of FIG. 1.

### DESCRIPTION OF THE INVENTION

Illustrative embodiments and exemplary applications will now be described with reference to the accompanying drawings to disclose the advantageous teachings of the present invention.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

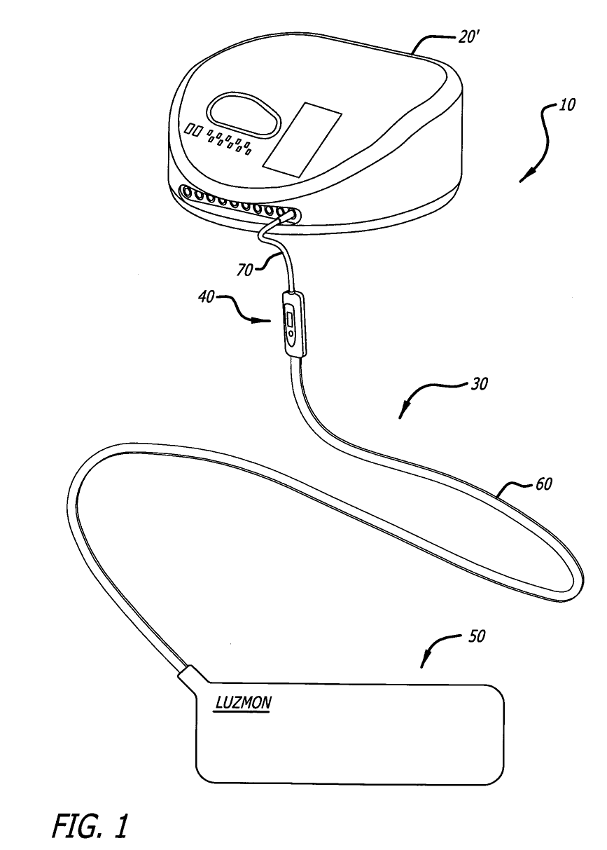

FIG. 1 is a simplified perspective view of a thermostimulation system implemented in accordance with an illustrative embodiment of the present teachings. As shown in FIG. 1, the system 10 includes a conventional thermostimulation console 20′ with, in accordance with the present teachings, a plurality of novel thermostimulation pad assemblies 30 electrically coupled thereto. Each pad assembly 30 includes a novel inline control system 40 and an associated multilayer injection molded dual function (heat and stimulation) pad 50 of unique design and construction with integrated sensor in accordance with the present teachings. Each control system 40 is connected to an associated pad 50 via a cable 60.

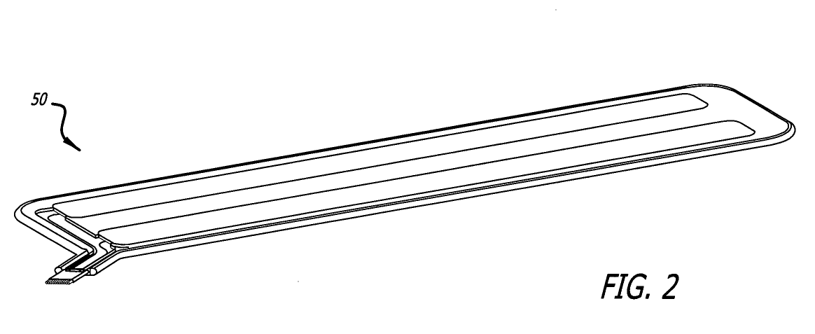

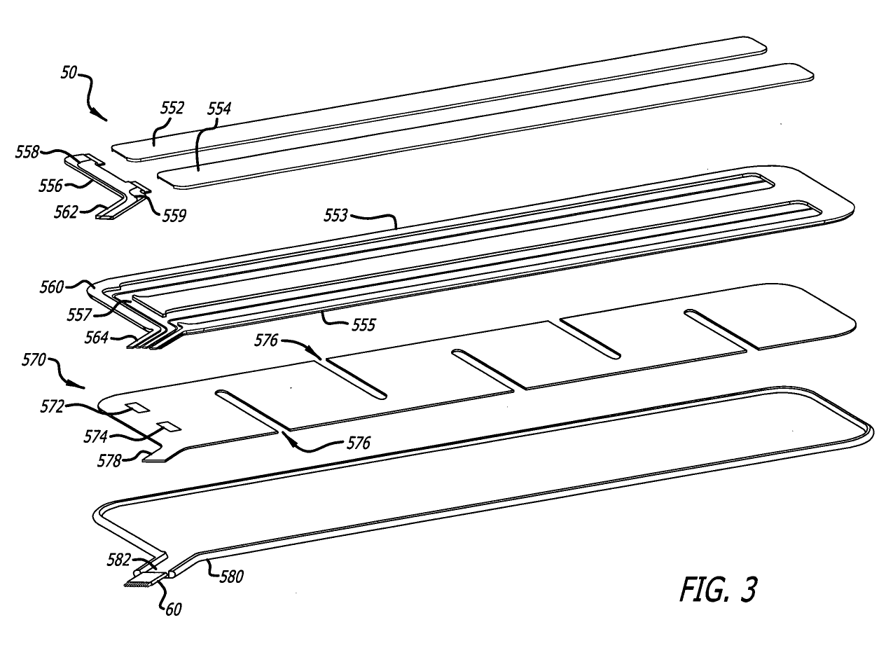

FIG. 2 shows a perspective bottom view of the pad 50 of FIG. 1. FIG. 3 is an exploded upside down view of a portion of the pad 50 of FIG. 1 in disassembled relation. As shown in FIGS. 2 and 3, the pad 50 includes first and second elongate substantially parallel conductive strips 552 and 554. In the illustrative embodiment, each conductive strip has a Shore hardness of 50—i.e. medical grade (USB Class 6) ten percent (10%) carbon loaded silicone. In the illustrative embodiment, the strips are 51.5 millimeters (mm) wide, 521 mm in length and 1.85 mm thick. Those of ordinary skill in the art will appreciate that the present teachings are not limited to the dimensions of the illustrative embodiment.

In the best mode, a polymer connector 556 is coupled to one end of the first and second strips 552 and 554 and serves as an end piece therefor and the second end of each strip is free. In the illustrative embodiment, the connector 556 is fabricated of Shore 40A silicone and serves as an insulator and support for wires 558 and 559 that provide a connection to the strips 552 and 554 respectively. In practice, one of the strips is powered a positive contact and the other provides a negative contact.

The two strips 552 and 554 are molded and then the end piece 556 is molded separately. These pieces are glued together with the wires 558 and 559 and placed back into a mold and the next layer 560 is over-molded over the assembly to provide a single molded piece consisting of the strips 552, 554, end piece 556, wires 558 and 559, and layer 560. In the preferred embodiment, the over-layer 560 is made of medical grade Shore 40A polymer or other material suitable for a particular application. Note the grooves 553 and 555 and recess 557 within the over-layer adapted to receive and seat the strips 552 and 554 and the end piece 556 respectively.

As shown in FIG. 3, a heating element 570 is provided over the layer 560. In the best mode, the heating element 570 is implemented as a built in wire matrix and is held in place with a layer of silicone 580. First and second temperature sensors 572 and 574 are mounted in the heating element 570, One is a live sensor measuring temperature and feeding this information back to the control box and the second is a back up should the first sensor fail. In the illustrative embodiment, each temperature sensor is implemented as a conventional 1 kilo-ohm RTD (resistance temperature detector). As noted in the article “Resistance thermometer” published Aug. 26, 2009, in the Wikipedia encyclopedia at http://en.wikipedia.org/wiki/Resistance_thermometer, Resistance thermometers, also called resistance temperature detectors or resistive thermal devices (RTDs), are temperature sensors that exploit the predictable change in electrical resistance of some materials with changing temperature. As they are almost invariably made of platinum, they are often called platinum resistance thermometers (PRTs). There are two broad categories, “film” and “wire-wound” types. Film thermometers have a layer of platinum on a substrate; the layer may be extremely thin, perhaps one micrometer. Advantages of this type are relatively low cost and fast response. In the best mode of practicing the present invention, film type RTDs are employed. Such sensors may be purchased from a number of manufacturers such as Minco Products of Minneapolis, Minn. (See www.minco.com.) However, those of ordinary skill in the art will appreciate that the present invention is not limited thereto. Other techniques for sensing temperature may be used without departing from the scope of the present teachings.

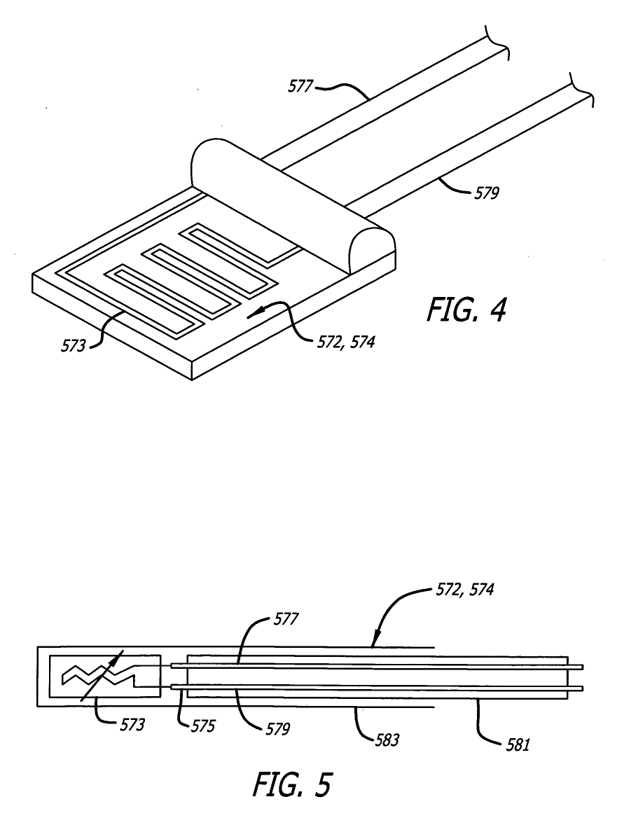

FIG. 4 is a magnified perspective view of a heat sensor implemented as a conventional resistance temperature detector (RTD) as depicted FIG. 3. FIG. 5 is a sectional side view of an illustrative implementation of the RTD sensor depicted in FIG. 4. As shown in FIGS. 4 and 5, the RTD 572, 574 includes a resistance thermometer 573 with connection leads 577 and 579 mounted within an insulator 581 disposed within a sheath 583. In the illustrative embodiment, the insulator 581 and the sheath 583 may be eliminated inasmuch as each RTD temperature sensor is mounted within the heating element 570 as shown herein.

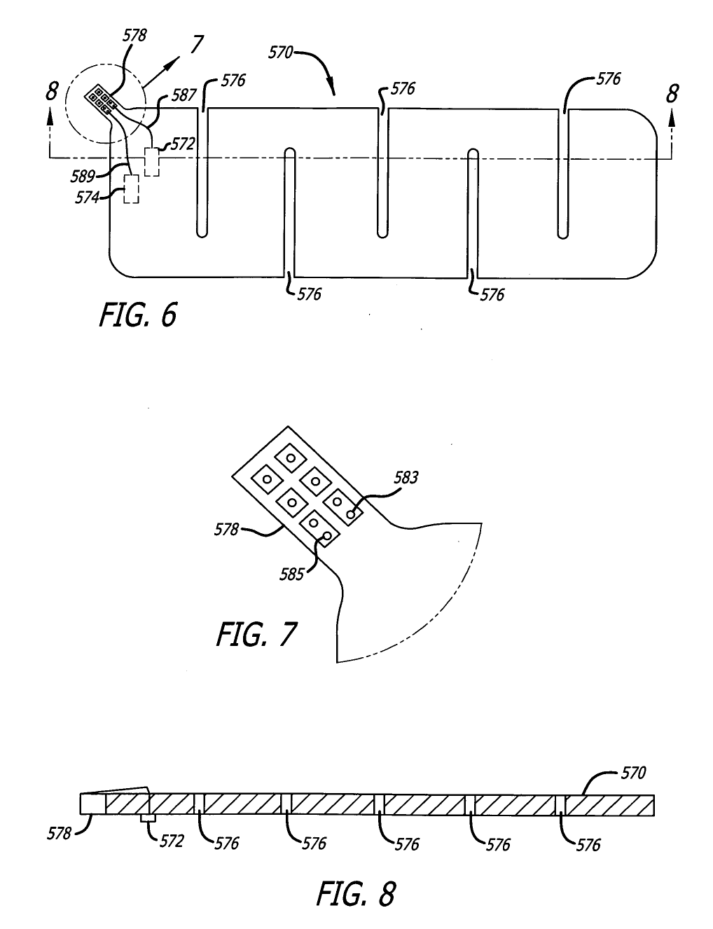

FIG. 6 is a top plan view of the heating element 570 of the illustrative embodiment of the pad of FIG. 1. In the illustrative embodiment, the heating element is a wire matrix bonded in silicone with a thickness of 0.75 mm, over the majority of the surface apart from where the RTDs are mounted, and is rated at 400 watts per square meter using 24 volts alternating current. Note the provision of slots 576 in the heating element 570. These slots serve to improve flexibility in all planes of the element. Those of ordinary skill in the relevant art will appreciate that the present invention is not limited to the design and/or construction of the heating element and that the heating element embodiment disclosed herein is for the purpose of illustration only.

In the illustrative embodiment, as shown in the top plan view of FIG. 6 and the magnified view of a portion thereof in FIG. 7, the extension 578 of the heating element 570 has a number of solder connections to facilitate electrical connection of the heating element 570 to the cable 60. A set of connections 583 and 585 provide a connection to the temperature sensors 572 and 574 via wire pairs 587 and 589 respectively.

FIG. 8 is a sectional side view of the pad of FIG. 1. As shown in FIGS. 3 and 8, the extension tab 578 is adapted to be received within a strain relief grommet 582 in the heater over-layer 580 along with the extensions 562 of the end piece 556 and 564 of the layer 560. In the illustrative embodiment, the grommet does not come into contact with the extension 578. The grommet 582 receives the flat cable 60 which is then stripped back and the associated wires are connected to the various solder pads on the extension 578. In the illustrative embodiment, the heater over-layer 580 is Shore 40A medical grade silicone in construction. Nonetheless, as noted above, it should be noted that the present invention is not limited to any particular material or hardness.

Each pad is assembled in a number of sections. Each pad is assembled from the stimulation side. In the best mode, the structure of the pad 50 is based on a multi-step injection molding process, with over-molding of the various layers to build up the base of the pad to the complete pad thickness and embed and encapsulate the various components within it, such as the electrostimulation wires and heating element. The final step is to insert and bond the top lid of the pad into the assembled structure. The steps of the injection molding process include moulding of the stimulation strips, over moulding of the stimulation strips to encapsulate the stimulation wires to create the patient facing surface of the pad and the moulding of the lid of the pad 580 which encapsulates the heating element and creates the upper facing surface of the pad and seals in the flat cable and grommet.

Hence, in accordance with the present teachings, the strips 552 and 554 and the layers 560, 570 and 580, temperature sensors 572 and 574 and the grommet 582 are molded into a single unitary multilayer injection molded dual function (heat and electrostimulation) construction.

Those skilled in the art will appreciate that the present invention is not limited to the materials utilized in the fabrication of the illustrative embodiment. Other materials may be used without departing from the scope of the present teachings.

The present invention thus enables new thermal and thermostimulation methods and protocols by which the electrical stimulation and/or the heating current supplied to a thermo and/or thermostimulation pad are adjusted based on the temperature sensed by the pad per se.

Thus, the present invention has been described herein with reference to a particular embodiment for a particular application. Those having ordinary skill in the art and access to the present teachings will recognize additional modifications, applications and embodiments within the scope thereof.

It is therefore intended by the appended claims to cover any and all such applications, modifications and embodiments within the scope of the present invention.

Accordingly,

What is claimed is:

1. A therapeutic pad comprising:
a heating element and
a temperature sensor operationally coupled to sense thermal energy within said pad in close proximity to said heating element.

2. The invention of claim 1 wherein said temperature sensor is a resistance thermal detector.

3. The invention of claim 2 wherein said temperature sensor is a film type resistance thermal detector.

4. The invention of claim 1 further including a second temperature sensor.

5. The invention of claim 1 including at least one contact for providing electrical stimulation.

6. The invention of claim 5 wherein said contact is disposed in a parallel relation with said heating element.

7. The invention of claim 5 including a second contact for providing electrical stimulation.

8. The invention of claim 7 wherein said heating element, said first and second electrical contacts and said sensor are encapsulated in a single unitary multilayer injection molded construction.

9. The invention of claim 1 wherein said heating element is a wire matrix.

10. The invention of claim 1 wherein said heating element and said sensor are encapsulated in a single unitary multilayer injection molded construction.

11. A therapeutic pad comprising:
first and second elongate electrical contacts;
an elongate heating element disposed in parallel relation with said contacts and
first and second temperature sensors operationally coupled to sense thermal energy within said pad in close proximity to said heating element, each of said temperature sensors being a film type resistance thermal detector sensor, wherein said heating element and said sensor are encapsulated in a single unitary multilayer injection molded construction.

12. The invention of claim 11 wherein said heating element is a wire matrix.

13. A thermostimulation method including the steps of:
providing a pad with first and second elongate electrical contacts; an elongate heating element disposed in parallel relation with said contacts and first and second temperature sensors operationally coupled to sense thermal energy within said pad in close proximity to said heating element, each of said temperature sensors being a film type resistance thermal detector sensor, wherein said heating element and said sensor are encapsulated in a single unitary multilayer injection molded construction;
providing stimulation and heating current to said pad; and
measuring the temperature detected by said sensors and adjusting the stimulation current and/or heating current in response to said detected temperature.
